(19) United States
(12) Patent Application Publication
(10) Pub. No.: US 2024/0374414 A1
(43) Pub. Date: Nov. 14, 2024

(54) FLUID COLLECTION CONTAINER LINER ASSEMBLY, AND RELATED SYSTEMS AND METHODS

(71) Applicant: PUREWICK CORPORATION, Covington, GA (US)

(72) Inventors: Tamika Richardson, Schenectady, NY (US); Padraig Fogarty, Ballina-Killaloe (IE); Anthony Garvey, Listowel (IE); Meghan Caracciola, Washington, DC (US)

(21) Appl. No.: 18/690,392

(22) PCT Filed: Sep. 21, 2022
(86) PCT No.: PCT/US2022/044212

§ 371 Date: Mar. 8, 2024

Related U.S. Application Data
(60) Provisional application No. 63/246,972, filed on Sep. 22, 2021.

(51) Int. Cl. A61F 5/455; A61F 5/44; A61F 5/441

(52) U.S. Cl. CPC A61F 5/4556; A61F 5/4405; A61F 5/441

(57) ABSTRACT

Examples relate to devices, systems, and methods for fluid collection. A fluid collection system may include a fluid collection device configured to collect fluid discharged from a user, a fluid collection container configured to receive the fluid from the fluid collection device, a pump in fluid communication with the fluid collection container configured to pull an at least partial vacuum on an interior region of the fluid collection container effective to draw fluid from the fluid collection device into the fluid collection container. The fluid collection container is a rectangular shape.

### CROSS-REFERENCE TO RELATED APPLICATIONS

This application claims priority to U.S. Provisional Patent application No. 63/246,972 filed on Sep. 22, 2021, entitled FLUID COLLECTION CONTAINER WITH DRAINAGE SPOUT AND RELATED SYSTEMS AND METHODS, the disclosure of which is incorporated by reference herein, in its entirety.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to fluid collection systems and methods of using fluid collection systems. In an embodiment, a fluid collection system may include a fluid collection device configured to collect fluid discharged from a user, a fluid collection container configured to receive the fluid from the fluid collection device, and a pump in fluid communication with the fluid collection container. The pump may be in fluid communication with the fluid collection container and configured to pull an at least partial vacuum on an interior region of the fluid collection container effective to draw fluid from a fluid collection device into the fluid collection container. The fluid collection container may be a rectangular shape.

In an embodiment, a portable fluid collection system may include a fluid collection device, a first tube in fluid communication with the fluid collection device, a fluid collection container including a rectangular shape, a pump in fluid communication with the fluid collection container, a power supply operatively coupled to the pump, and a drainage tube configured to couple to the fluid collection container. The fluid collection device may be configured to be positioned at least proximate to a urethra of a user. The pump may be configured to pull an at least partial vacuum on an interior region of the fluid collection container effective to draw fluid from the fluid collection device through the first tube into the fluid collection container.

In an embodiment, a method of draining a fluid collection system includes de-energizing the fluid collection system. The fluid collection system can include a fluid collection container having a drainage port and the method can further include attaching a drainage tube to the drainage port. In some embodiments, the drainage tube includes a valve. The method also includes opening a vent on the fluid collection container and opening the valve to discharge a fluid from the fluid collection system.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a portable fluid collection system, according to an embodiment.

FIG. 1B is a front isometric view of a fluid collection system, according to an embodiment.

FIG. 2 is an isometric view of a fluid collection container, according to an embodiment.

FIG. 3 is an isometric view of an elbow connector, according to an embodiment.

FIG. 4A is a front isometric view of a portable carrying case for a fluid collection system, according to an embodiment.

FIG. 4B is a cross sectional view of a portable fluid collection system and carrying case, according to an embodiment.

FIG. 5 is a flow diagram of a method for draining a fluid collection system, according to an embodiment.

### DETAILED DESCRIPTION

Embodiments disclosed herein are related to fluid collection containers and systems including liner assemblies, and related methods. The devices and systems disclosed herein are configured to collect fluids from an individual. The fluids collected by the fluid collection devices may include at least one of urine, vaginal discharge, penile discharge, reproductive fluids, blood, sweat, or other bodily fluids.

In conventional urine collection systems, the urine is collected in a urine collection container that can then be emptied and washed. Emptying and washing the urine collection container after every use can be an unpleasant, tedious, time consuming, and frustrating chore for the user and/or a caregiver.

To improve conventional urine collection systems, fluid collection containers described herein may include a drainage tube in some embodiments to make removal of the urine much simpler and easier to perform, thereby minimizing the inconvenience of cleaning the system and also minimizing odors common to conventional urine collection systems. In some embodiments, the fluid collection system can also include a fluid collection container that is rectangular shaped to make the urine collection system more portable. Portable can be defined more able to be easily carried or moved, being of a lighter and smaller version than fluid collection systems that include large capacity of urine collection. In other words, the portable carrying case can be carryable by a human, such as a caretaker or a patient.

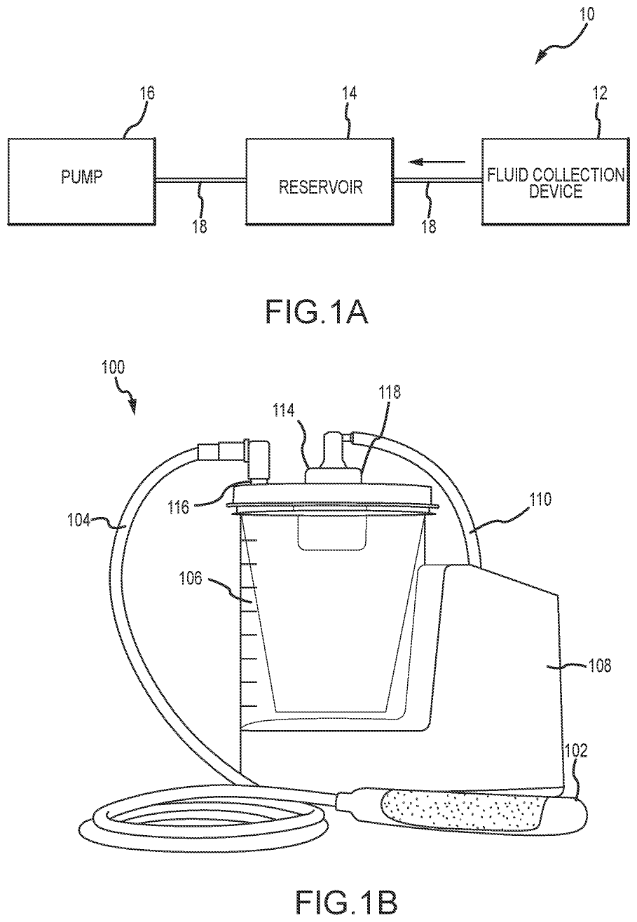

FIG. 1A is a block diagram of a fluid collection system 10, according to an embodiment. The fluid collection system 10 may be included in embodiments of fluid collection systems described herein. The system 10 includes a fluid collection device 12 (e.g., any of the urine collection devices disclosed herein), a reservoir (or fluid collection container) 14, and a pump 16 (or vacuum source). The fluid collection device 12, the fluid collection container 14, and the pump 16 may be fluidly coupled to each other via one or more tubes 18. For example, fluid collection device 12 may be operably coupled to one or more of the fluid collection container 14 or the pump 16 via the tube 18. In some embodiments, the pump 16 may be secured directly to the fluid collection container 14. Fluid (e.g., urine or other bodily fluids) collected in the fluid collection device 12 may be removed from the fluid collection device 12 via the tube 18 secured to the fluid collection device 12. Suction force may be introduced into the chamber of the fluid collection device 12 via the inlet of the tube 18 responsive to suction (e.g., vacuum) force applied at the outlet of the tube 18.

The suction force may be applied to the outlet of the tube 18 by the pump 16 either directly or indirectly. The suction force may be applied indirectly via the fluid collection container 14. For example, the outlet of the tube 18 may be disposed within or fluidly coupled to an interior region of the fluid collection container 14 and an additional tube 18 may extend from the fluid collection container 14 to the pump 16. Accordingly, the pump 16 may apply suction to the fluid collection device 12 via the fluid collection container 14. The suction force may be applied directly via the pump 16. For example, the outlet of the tube 18 may be disposed within the pump 16. An additional tube 18 may extend from the pump 16 to a point outside of the fluid collection device 12, such as to the fluid collection container 14. In such examples, the pump 16 may be disposed between the fluid collection device 12 and the fluid collection container 14.

The fluid collection container 14 is sized and shaped to retain a fluid therein. The fluid collection container 14 may include a bag (e.g., drainage bag), a bottle, a canister, or a cup (e.g., collection jar), or any other enclosed container for storing bodily fluid(s) such as urine. In some examples, the tube 18 may extend from the fluid collection device 12 and attach to the fluid collection container 14 at a first point therein. An additional tube 18 may attach to the fluid collection container 14 at a second point thereon and may extend and attach to the pump 16. Accordingly, a vacuum (e.g., suction) may be drawn through fluid collection device 12 via the fluid collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

FIG. 1B shows an example of a fluid collection system 100, according to an embodiment. The fluid collection system 100 may include a fluid collection device 102, a first tube 104 in fluid communication with an interior region of the fluid collection device 102, a fluid collection container 106, and a pump 108. The fluid collection device 102 may be configured to be positioned at least proximate to a urethra of a user. While the fluid collection device 102 shown in FIG. 1B includes a female urine collection device, the fluid collection device 102 may instead include a male urine collection device. PCT International Application No. PCT/US2019/029616, for example, describes various embodiments of both male and female fluid collection devices, the disclosure of which are incorporated in their entirety by this reference. Moreover, the fluid collection device 102 may be interchangeable in the fluid collection system 100 between different types, varieties, and sizes of male or female urine collection devices. Generally, the fluid collection device 102 may include a surface sized to be positioned proximate or adjacent to the urethra and configured to wick urine or other fluids away from the user. Urine or other fluids may be wicked from the surface to a reservoir in the fluid collection device 102.

The fluid collection system 100 also includes the first tube 104 in fluid communication with an interior region (e.g. reservoir) of the fluid collection device 102 and the interior region of the fluid collection container 106. The first tube 104 may be positioned between the fluid collection device 102 and the fluid collection container 106. The fluid collection system 100 also may include a second tube 110 providing fluid communication between the pump 108 and the interior region of the fluid collection container 106. In some embodiments, the pump 108 may be secured directly to the fluid collection container 106, and the second tube 110 may be absent from the fluid collection system 100. The tubes 104 and 110 may include a flexible tube.

The fluid collection container 106 may be reusable and dishwasher safe, and may include a durable plastic material such as polycarbonate or glass. In some embodiments, the fluid collection container 106 may be disposable. The fluid collection system 100 may be configured to accommodate different volumetric sizes of fluid collection containers 106. In some embodiments, the fluid collection container 106 may include at least one of a volume having 500 mL. 800 mL, 1000 mL, or 2000 mL. In some embodiments, the fluid collection container 106 includes a capacity from about 800 mL to about 1000 mL. Except for the ports and/or adapters for attachment of the tubes 104, 110, the fluid collection container 106 may be sealed and airtight such that the urine collected in the fluid collection container 106 does not leak or spill.

In some embodiments, the fluid collection container 106 may include a rectangular shape. The rectangular shape may make the fluid collection system 100 more portable. The rectangular shape may make the fluid collection container 106 easier to drain and/or clean, in some embodiments. In some embodiments, the fluid collection container can include a cubic shape. The fluid collection system 100 may be made to be disposed in a carrying case or bag, such as a backpack and/or shoulder bag.

The pump 108 is in fluid communication with the interior region of the fluid collection container 106 and is configured to pull a vacuum on the interior region of the fluid collection container 106 effective to draw the urine from the fluid collection device 102 through the first tube 104 into the fluid collection container 106. The pump may be configured to draw urine from the fluid collection device 102 by applying at least a partial suction force to a reservoir located within the fluid collection device 102. The pump 108 may be secured directly to the fluid collection container 106, or the second tube 110 may fluidly couple the pump 108 with the interior region of the fluid collection container 106. The pump 108 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 108 may provide a vacuum or suction to remove fluid from the fluid collection device 102. In some examples, the pump 108 may be powered by one or more batteries or other power sources. The pump 108 may be included in a housing that also supports the fluid collection container 106. In some embodiments, the housing may be rectangular shaped. In some examples, the pump 108 and/or housing may be sized and shaped to fit within a portable carrying case. The pump 108 may include one or more of a switch, a button, a plug, a remote, or any other device suitable to activate the pump 108.

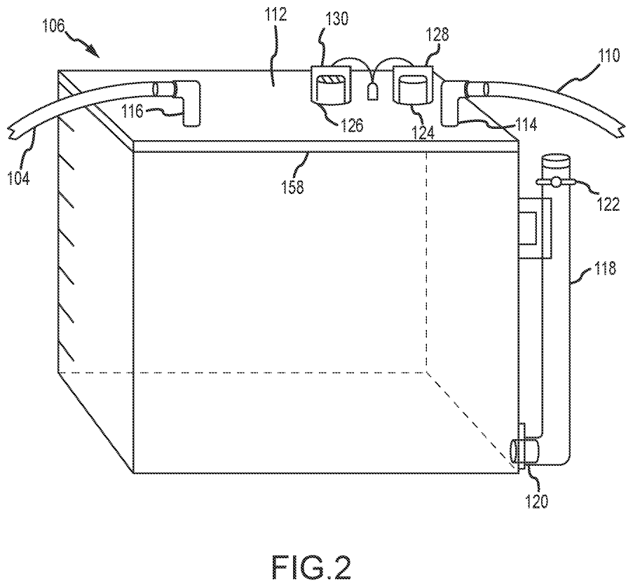

FIG. 2 illustrates an embodiment of fluid collection container 106. The fluid collection container 106 may include a durable plastic canister and may include a capacity from about 800 mL to about 1000 mL. The fluid collection container 106 may exhibit a rectangular shape to allow the container 106 to securely and discretely fit within a portable carrying case. The fluid collection container 106 may be reusable and dishwasher safe, and may include a generally rigid material such as polycarbonate, plastic, rubber, metal, glass, combinations thereof, or any other suitable materials. In other embodiments, the fluid collection container 106 may be disposable and biodegradable.

In some embodiments, the fluid collection container 106 may include a drain tube 118 to remove fluid from the fluid collection container 106. Fluid collected in the fluid collection container 106 may be emptied through the drain tube 118. The drain tube 118 may be coupled to a drain port 120. In some embodiments, the drain port 120 may be located in a bottom corner of the fluid collection container 106. In some embodiments, the drain port 120 may activate when the drain tube 118 couples to the drain port 120. For example, the drain port 120 may include a valve that opens when the drain tube 118 is coupled to the drain port 120. In some embodiments, the drain port 120 may include a split septum device that opens when the drain tube 118 is inserted. In other embodiments, the drain port 120 may be a one-way valve that is activated open when the drain tube 118 is connected. In some embodiments, the pump 108 may de-energize when the drain tube 118 is coupled to the drain port 120 and/or the fluid collection container 106. In some embodiments, the drain tube 118 may include a valve 122 that may be opened to empty the fluid collection container 104 directly or a pump may be coupled to the fluid collection container 104 and configured to apply a pressure to the fluid for more rapid disposal. The valve 122 may include a clamp, a ball valve, a butterfly valve, or any suitable valve.

In some embodiments, the fluid collection container 106 includes a lid 112 and a drain spout 124 and vent spout 126 coupled to the lid 112. In some embodiments, the lid 112 further includes a collection port 116 for the first tube 104 and a vacuum port 114 for the second tube 110. The drain spout 124 may include a first cover 128 that covers the drain spout 124 when not in use. The first cover 128 may be connected to a second cover 130 for the vent spout 126 and configured such that the second cover 130 is removed and the vent spout 126 is open when the fluid is dispensed through the drain tube 118, to aid in proper air flow as the fluid is removed. In some embodiments, the lid 112 may include additional ports or adapters than those shown in FIG. 2.

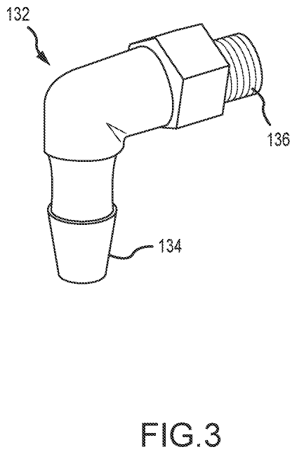

FIG. 3 is an isometric view of an elbow connector 132, according to an embodiment. The elbow connector 132 can be included in the fluid collection system 100 as a connector to the lid 112, in some embodiments. In some embodiments, the elbow connector 132 may include a tapered opening 134. The tapered opening 134 may allow tubing of different sizes to be coupled to the elbow connector 132. For example, a type of fluid collection device (e.g. fluid collection device 102) may include a tubing having a first diameter and the second tube 116 may include a tubing having a second diameter. The first diameter and the second diameter can be different. The tapered openings 134 of the elbow connectors 132 can connect with either diameter tubing without replacing the elbow connector 132. The vacuum port 114 and/or the collection port 116 may include the elbow connector 132. The elbow connector 132 can further include a connector opening 136 configured to connect to the lid 112 of the fluid collection container 106 for example. In some embodiments, the connector opening 136 can include threads or any other suitable fastener.

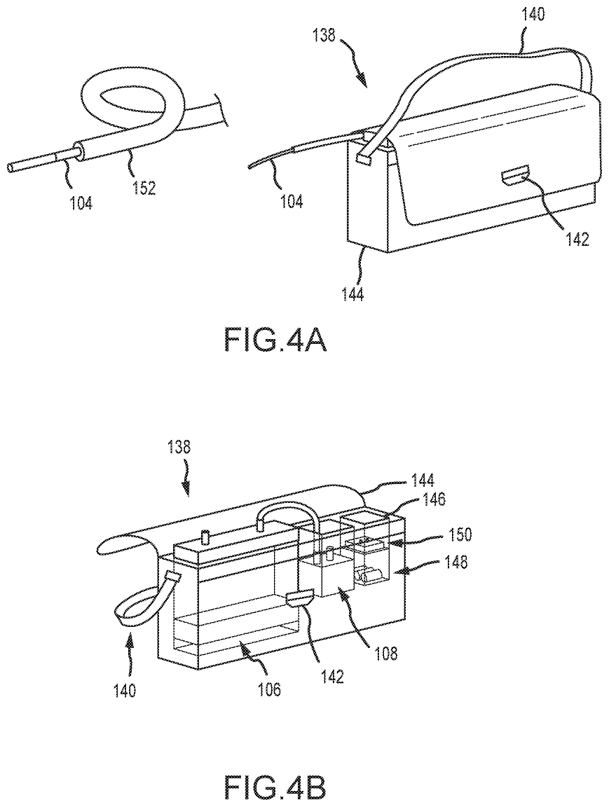

Referring now to FIGS. 4A-4B, the fluid collection system 100 may include a portable carrying case 138 that may be worn by a user and/or a caregiver. In some embodiments, the portable carrying case 138 may include one or more straps 140 to couple the portable carrying case 138 to at least one of the user, one or more wheelchair handles, a bed, or a table. In some embodiments, an outer surface of the portable carrying case 138 may include a fastener 142. The fastener may include a buckle, Velcro, or other suitable fastener configured to keep the portable carrying case 138 closed. The portable carrying case 138 may be a generally rectangular shape with a flat bottom for stability and for being able to fit underneath a wheelchair and/or coupled to a back of the wheelchair. The one or more straps 140 may be configured to be worn on a shoulder or to detachably secure, mount, or hang from a stationary or mobile structure (e.g., a wheelchair) and support the fluid collection system 100. In some embodiments, the portable carrying case 138 may include at least one of a backpack or shoulder bag. In some embodiments, the portable carrying case 138 may include a durable outer cover 144 and/or shell.

In some embodiments, the portable carrying case 138 may include one or more compartments 146, each compartment being able to be accessed separately. In some embodiments, the compartments 146 can be disposed within sound-dampening material such as a foam or other suitable material to minimize the sound of the pump 108 operations and/or fluid sloshing within the fluid collection container 106. The portable carrying case 138 may be closeable for discretion of the fluid collection system 100 and may be used to hold other items securely. For example, when the drain tube 118 is not being used, the drain tube 118 may be stored within the portable carrying case 138. The portable carrying case 138 may be sized and dimensioned to hold at least the fluid collection container 106, a pump 108, and a battery 148, and/or a battery charger 150 (shown in FIG. 4B) therein.

In some embodiments, one or more of the tubes 104, 110, 118 can include a fabric sleeve 152. For example, the first tube 104 may be configured to be disposed within the fabric sleeve 152. The fabric sleeve 152 can include an opaque covering to improve discretion for the user. The fabric sleeve 152 can conceal the flow of urine or other fluid through the first tube 104 as the fluid is drawn from the fluid collection device 102 to the fluid collection container 106.

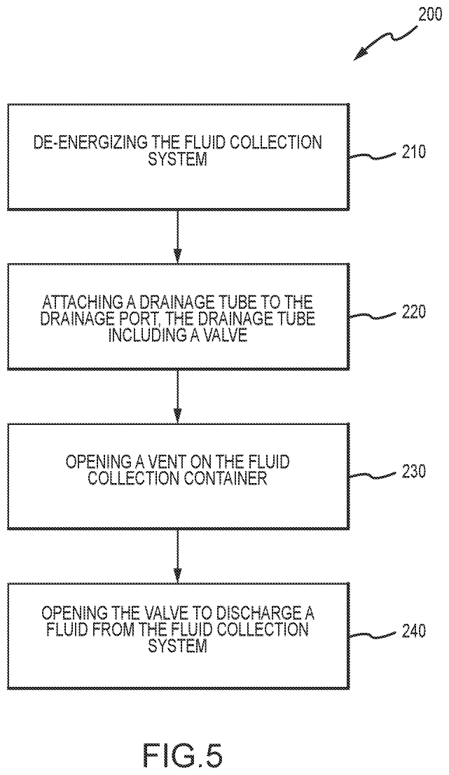

FIG. 5 is a flow diagram of a method 200 for draining a fluid collection system (e.g. fluid collection system 100), according to an embodiment. The method 200 includes an act 210 of de-energizing the fluid collection system. De-energizing the fluid collection system ensures the pressure within the fluid collection container is low such that the fluid drains from the fluid collection container. In some embodiments, the method 200 includes an act 220 of attaching a drainage tube to the drainage port. In some embodiments, the fluid collection system includes a fluid collection container having a drainage port. The drainage port can include a valve that opens when the drainage tube is attached to the drainage port. The valve within the drainage port may be a septum valve, a diaphragm valve, or other suitable valve.

In some embodiments, fluid collection system may de-energize when the drainage tube is attached to the drainage port. In some embodiments, the drainage tube includes a valve that the user can open and shut to control the flow of fluid through the drainage tube. In some embodiments, the valve includes a tube clamp. In other embodiments, the valve may include a butterfly valve, septum valve, etc.

In some embodiments, the method 200 includes an act 230 of opening a vent on the fluid collection container. In some embodiments, the vent can be included on a lid of the fluid collection container. The lid itself, when removed, can be an open vent in some embodiments. The vent can be opened to ensure the pressure within the container allows for flow through the drainage tube and the container is vented to ensure proper flow. In some embodiments, the method 200 includes an act 240 of opening the valve to discharge the fluid from the fluid collection system. The opening of the drainage tube valve ensures the user maintainers control of the direction and flowrate of the fluid exiting the drainage tube. For example, an end of the drainage tube can be placed into a toilet bowl and the user can open the valve on the drainage tube to drain the fluid into the toilet bowl. In some embodiments, after the fluid has been drained from the fluid collection container, the valve is shut and the fluid collection system is restored to its original state. To restore the system, in some embodiments, the vent is shut and the drainage tube can be disconnected from the drainage port. The fluid collection system can then be re-energized to draw fluid from the fluid collection device into the fluid collection container.

The acts of the method 200 described above are for illustrative purposes. For example, the acts of the method 200 can be performed in different orders, split into multiple acts, modified, supplemented, or combined. In an embodiment, one or more of the act of the method 200 can be omitted. Any of the acts of the method 200 can include using any of the fluid collection systems and/or liner assemblies disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A fluid collection system, comprising:
a fluid collection device configured to collect fluid discharged from a user;
a fluid collection container configured to receive the fluid from the fluid collection device, wherein the fluid collection container is a rectangular shape; and
a pump in fluid communication with the fluid collection container configured to pull an at least partial vacuum on an interior region of the fluid collection container effective to draw fluid from a fluid collection device into the fluid collection container;
wherein the fluid collection container includes a drainage port and a drainage tube, wherein the drainage port activates a valve that opens when the drainage tube is coupled to the drainage port.

2. The fluid collection system of claim 1, wherein the rectangular shape includes a cubic shape.

3. The fluid collection system of claim 1, wherein the fluid collection container is sized and dimensioned to be disposed within a portable carrying case.

4. The fluid collection system of claim 1, wherein the fluid collection container includes a durable plastic.

5. (canceled)

6. (canceled)

7. The fluid collection system of claim 1, wherein the drainage tube includes a valve that opens to drain the fluid collection container.

8. (canceled)

9. The fluid collection system of claim 1, wherein the fluid collection container includes a capacity from about 800 mL to about 1000 mL.

10. A portable fluid collection system, comprising:
a fluid collection device configured to be positioned at least proximate to a urethra of a user;
a first tube in fluid communication with the fluid collection device;
a fluid collection container exhibiting a rectangular shape;
a pump in fluid communication with the fluid collection container through a second tube, wherein the pump is configured to pull an at least partial vacuum on an interior region of the fluid collection container effective to draw fluid from the fluid collection device through the first tube into the fluid collection container;
a power supply operatively coupled to the pump; and
a drainage tube configured to couple to the fluid collection container, wherein the pump de-energizes when the drainage tube is coupled to the fluid collection container.

11. The portable fluid collection system of claim 10, wherein the fluid collection system is sized and dimensioned to be disposed within a portable carrying case.

12. The portable fluid collection system of claim 10, wherein the first tube is configured to be disposed within a fabric sleeve.

13. The portable fluid collection system of claim 12, wherein at least a portion of the fabric sleeve includes an opaque covering.

14. (canceled)

15. The portable fluid collection system of claim 10, further comprising a lid for the fluid collection container, the lid including a dispensing spout and a vent.

16. The portable fluid collection system of claim 15, wherein the dispensing spout includes a first removable cover and the vent includes a second removable cover, wherein the first removable cover and the second removable cover are connected.

17. The portable fluid collection system of claim 15, wherein the lid includes a first connector coupled to the first tube and a second connector coupled to the second tube, wherein the first connector and the second connector include elbow connectors.

18. The portable fluid collection system of claim 17, wherein the elbow connectors include a tapered opening.

19. A method of draining a fluid collection system, the method comprising:
de-energizing the fluid collection system, wherein the fluid collection system includes a fluid collection container having a drainage port;
attaching a drainage tube to the drainage port, wherein the drainage port includes a first valve that opens when the drainage tube is attached to the drainage port and wherein the drainage tube includes a second valve;
opening a vent on the fluid collection container; and
opening the second valve to discharge a fluid from the fluid collection system.

20. (canceled)
